(19) United States
(12) Patent Application Publication
(10) Pub. No.: US 2004/0026287 A1
(43) Pub. Date: Feb. 12, 2004

(54) Moist eyeglass cleaner

(76) Inventor: Allan Tsai, Taipei (TW)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 10/212,037

(22) Filed: Aug. 6, 2002

(51) Int. Cl. G02C 13/00; A61B 17/06

(52) U.S. Cl. 206/440; 15/104.93; 15/214

(57) ABSTRACT

A moist eyeglass cleaner includes an impermeable reclosable handy packet, and a plurality of stacked moist paper sheets disposed in the packet. The packet includes a first wall having an open side formed with an adjacent pair of fixed parallel ribs, and a second wall having an open side formed with a fixed rib that is clamped between the ribs of the first wall so that the packet is reclosable.

### BACKGROUND OF THE INVENTION

1. Field of the Invention

The invention relates to an eyeglass cleaner, more particularly to a moist eyeglass cleaner.

2. Description of the Related Art

Referring to FIGS. 1 to 3, a conventional moist eyeglass cleaner comprises an impermeable handy packet 10 containing a single piece of multi-folded moist paper sheet 11. Since the volume of the packet 10 has to be small so as to be handy, the moist paper sheet 11 has to be half-folded 110 many times so as to fit into the packet 10. The packet 10 has one peripheral side formed with a cut-out portion 12 to facilitate tearing of the packet 10. In use, the packet 10 is torn along the cut-out portion 12, and the moist paper sheet 11 is removed from the packet 10 and is unfolded. When the moist paper sheet 11 is completely unfolded, it can be used to wipe eyeglasses. However, the conventional moist eyeglass cleaner has the following disadvantages:

1. Since only one moist paper sheet 11 is contained in the packet 10, the moist paper sheet 11 and the packet 10 are both disposed after use, which is a waste and which easily causes environmental pollution.

2. It is inconvenient to carry the packet 10 in large quantities for multiple use because it will occupy a relatively large space in a bag.

3. Although the handy packet 10 is compact, the cost of the packet 10 is relatively high because a lot of materials are needed. Furthermore, a lot of manpower is wasted due to the multi-folding operation of the moist paper sheet 11.

### SUMMARY OF THE INVENTION

Therefore, the main object of the present invention is to provide a moist eyeglass cleaner that is capable of overcoming the aforementioned drawbacks of the prior art.

According to this invention, a moist eyeglass cleaner includes an impermeable reclosable handy packet and a plurality of stacked moist paper sheets that are disposed in the packet and that are adapted for cleaning an eyeglass. The packet includes a first wall having an open side formed with an adjacent pair of fixed parallel ribs, and a second wall having an open side formed with a fixed rib that is clamped between the ribs of the first wall so that the packet is reclosable.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of a conventional moist eyeglass cleaner;

FIG. 2 is an exploded perspective view of the conventional moist eyeglass cleaner in a state of use;

FIG. 3 is a perspective view of a moist paper sheet of the conventional moist eyeglass cleaner in an unfolded state;

FIG. 4 is an assembled perspective view of the preferred embodiment of a moist eyeglass cleaner according to the present invention;

FIG. 5 is an exploded perspective view of the preferred embodiment; and

FIG. 6 is a perspective view of a moist paper sheet of the preferred embodiment in a half-folded state.

### DETAILED DESCRIPTION OF THE PREFERRED EMBODIMENT

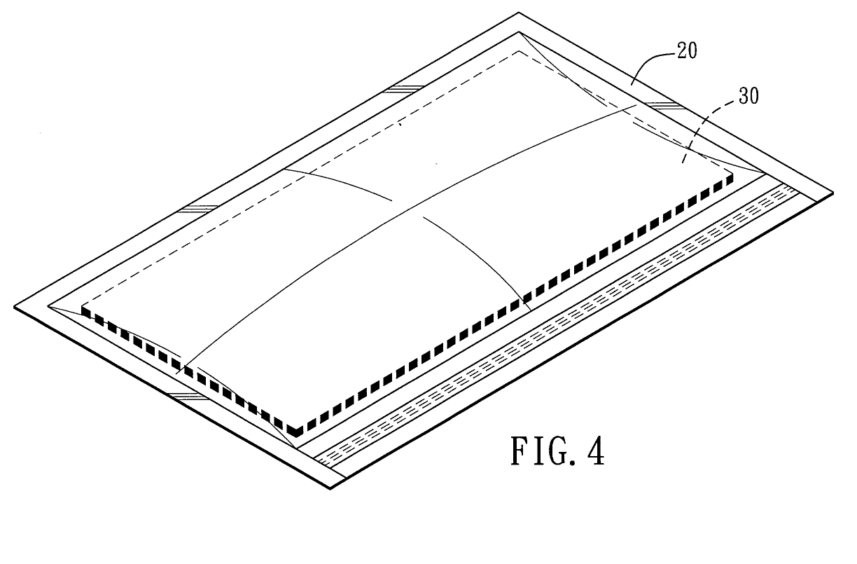

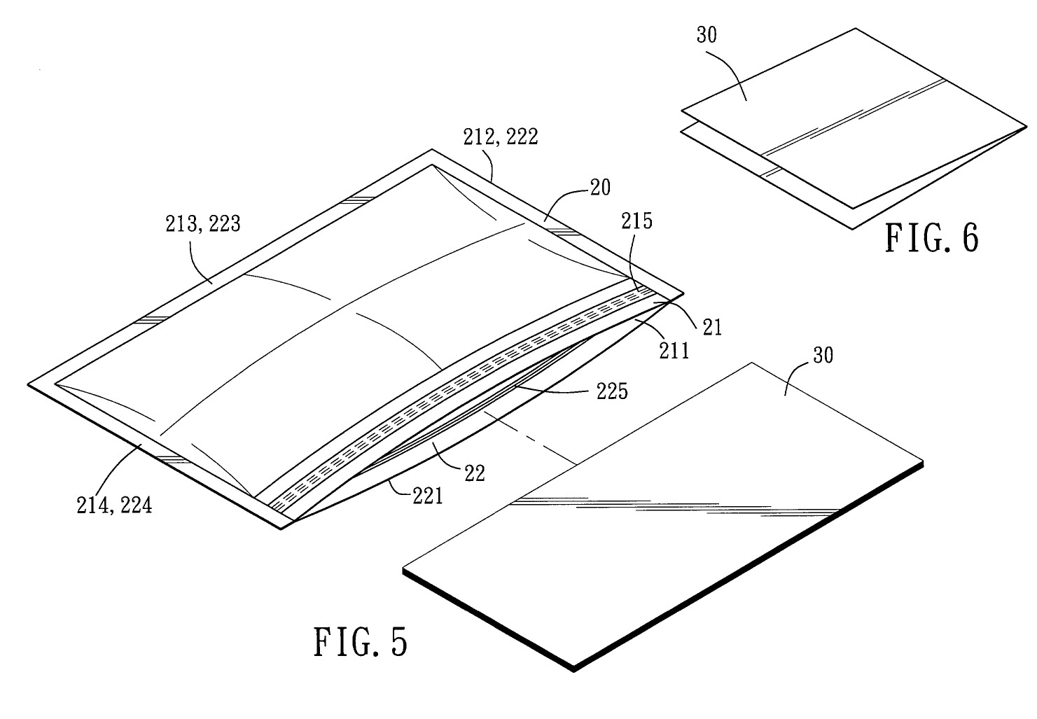

Referring to FIGS. 4 to 6, the preferred embodiment of a moist eyeglass cleaner according to the present invention is shown to comprise an impermeable reclosable handy packet 20, and a plurality of stacked moist paper sheets 30 that are disposed in the packet 20 and that are adapted for cleaning an eyeglass (not shown).

The packet 20 is made of an impermeable material, preferably aluminum foil. The packet 20 includes a rectangular first wall 21 and a rectangular second wall 22, each of which has an open side 211, 221 and three closed sides 212, 213, 214, 222, 223, 224. The first and second walls 21, 22 are interconnected fixedly along the closed sides 212, 213, 214, 222, 223, 224. The open side 211 of the first wall 21 is formed with an adjacent pair of fixed parallel ribs 215. The open side 221 of the second wall 22 is formed with a fixed rib 225 that is clamped between the ribs 215 of the first wall 21 so that the packet 20 is reclosable.

The moist paper sheets 30 are stacked and disposed in the packet 20. Each of the moist paper sheets 30 is soaked with eyeglass cleaning solution. Since the packet 20 is impermeable, and since the open sides 211, 221 of the packet 20 are sealed by the ribs 215, 225, the eyeglass cleaning solution is prevented from vaporizing.

In use, the open sides 211, 221 of the packet 20 are opened by separating the rib 225 of the second wall 22 from the ribs 215 of the first wall 21, after which one or more moist paper sheets 30 can be removed easily from the packet 20 for cleaning the eyeglass. The rib 225 of the second wall 22 is then pressed back between the ribs 215 of the first wall 21 so as to seal the open sides 211, 221 of the packet 20, thereby preventing the eyeglass cleaning solution from vaporizing. As such, the remaining moist paper sheets 30 are stored safely for subsequent use.

An appropriate size of the moist paper sheet 30 can be produced so that folding of the same is not necessary. After the moist paper sheet 30 is removed from the packet 20, it can be directly used to wipe the lens of the eyeglass. Alternatively, each of the moist paper sheets 30 can be fitted into a smaller, rectangular, impermeable reclosable handy packet (not shown) in a half-folded state, as shown in FIG. 6. The smaller packet is similar to the packet 20 in construction except that the size of the smaller packet is half of that of the packet 20. In this case, the packet 20 preferably contains about 15-30 pieces of the moist paper sheets 30, since a greater number of the paper sheets 30 would make the packet 20 bulge.

Some of the advantages of the moist eyeglass cleaner of the present invention can be summarized as follows:

1. Since one packet 20 contains many moist paper sheets 30, only the used single sheet is thrown away, thereby reducing waste and environmental pollution.

2. Since one packet 20 contains many moist paper sheets 30, it is very convenient to carry the packet 20 in a bag or in a pocket for multiple use.

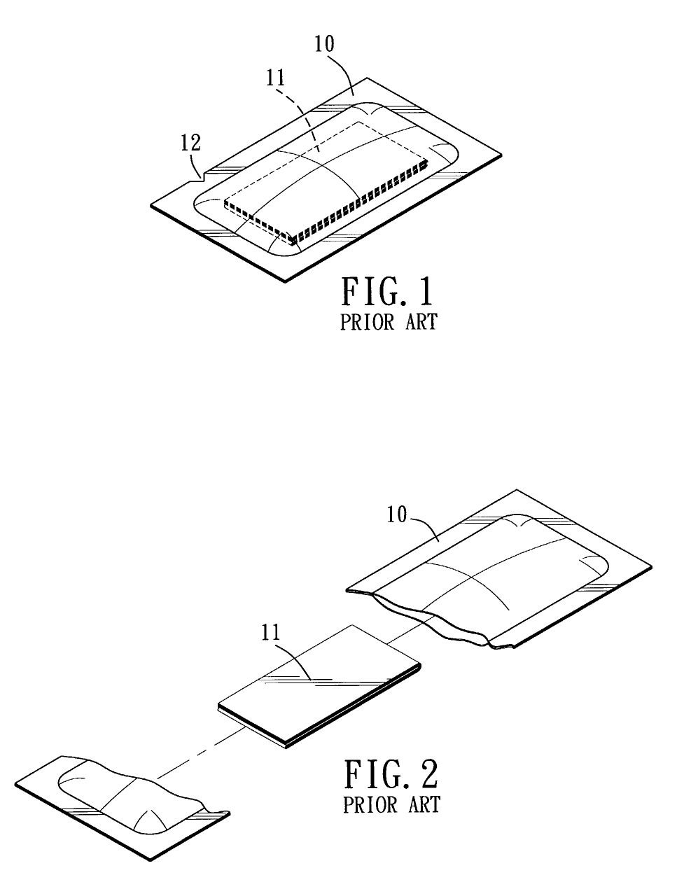

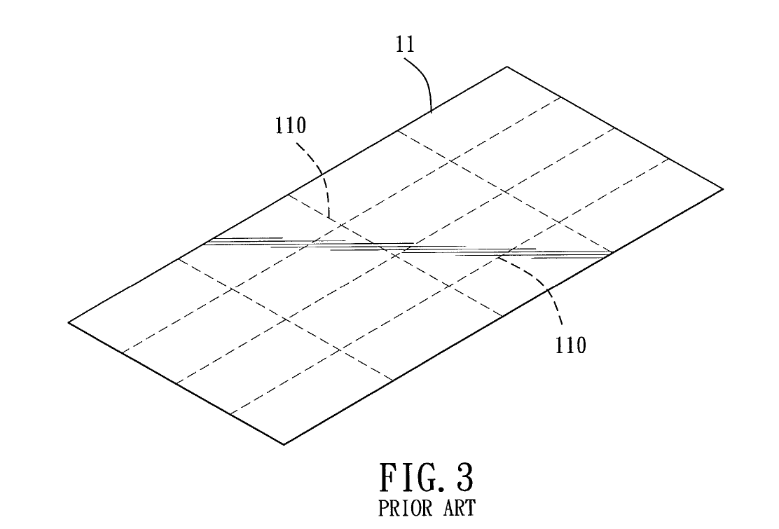

3. The price of the packet 20 is much cheaper, which is around one-third of that of the conventional moist eyeglass cleaner 10 (see FIGS. 1 and 2), because material requirement is reduced. Furthermore, packing is easy, which reduces waste of manpower.

4. Since the packet 20 is reclosable, after all the moist paper sheets 30 are used up, the packet 20 can be recycled for other purposes.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

I claim:

1. A moist eyeglass cleaner comprising an impermeable reclosable handy packet and a plurality of stacked moist paper sheets that are disposed in said packet and that are adapted for cleaning an eyeglass.

2. The moist eyeglass cleaner of claim 1, wherein each of said moist paper sheets in said packet is half-folded once, said packet containing about 15-30 pieces of said moist paper sheets.

3. The moist eyeglass cleaner of claim 1, wherein said packet includes a rectangular first wall and a rectangular second wall, each of which has an open side and three closed sides, said first and second walls being interconnected fixedly along said closed sides, said open side of said first wall being formed with an adjacent pair of fixed parallel ribs, said open side of said second wall being formed with a fixed rib that is clamped between said ribs of said first wall so that said packet is reclosable.
